(19) United States
(12) Patent Application Publication
(10) Pub. No.: US 2010/0322858 A1
(43) Pub. Date: Dec. 23, 2010

(54) Radiohaloimatinibs and Methods of Their Synthesis and Use in PET Imaging of Cancers

(75) Inventors: William Bornmann, Missouri City, TX (US); Mian Alauddin, Houston, TX (US); Juri Gelovani, Missouri City, TX (US); Pradip Ghosh, Houston, TX (US); Liwei Guo, Birmingham, AL (US); Dongmei Han, Houston, TX (US); David Maxwell, Pearland, TX (US); Uday Mukhopadhyay, Houston, TX (US); Zhenghong Peng, Missouri City, TX (US); Aleksandr Shavrin, Porter, TX (US); Yunming Ying, Houston, TX (US)

Correspondence Address: WILLIAMS, MORGAN & AMERSON, 10333 RICHMOND, SUITE 1100, HOUSTON, TX, 77042, US

(73) Assignee: BOARD OF REGENTS, THE UNIVERSITY OF TEXAS SYSTEM

(21) Appl. No.: 12/438,045

(22) PCT Filed: Aug. 22, 2007
(86) PCT No.: PCT/US07/76503

§ 371 Date: Sep. 8, 2010

Related U.S. Application Data
(60) Provisional application No. 60/823,324, filed on Aug. 23, 2006.

(51) Int. Cl. A61K 49/04; C07H 5/02

(52) U.S. Cl. 424/1.73; 536/122

(57) ABSTRACT

We disclose methods of synthesizing radiohalidated organic compounds and their use in positron emission tomography (PET) imaging of cancer cells.

### BACKGROUND OF THE INVENTION

The present invention relates generally to the field of radiolabeled markers for positron emission tomography (PET) imaging. More particularly, it concerns methods of synthesizing various radiolabeled markers and methods for their use.

A large number of lactose-binding proteins arc known, in both humans and other animals. Expression of particular lactose-binding proteins can vary in a number of cancers. For example, lectin is overexpressed in several types of tumors, including pancreatic carcinomas and colon carcinomas.

The ability to detect over- or underexpression of various lactose-binding proteins would be useful in detecting tumors and their extent and type.

### SUMMARY OF THE INVENTION

One embodiment of the present invention relates to a method of synthesizing an alkyl 2′-radiohalodeoxylactose, comprising treating an alkyl 2′-triflate deoxylactose with n-Bu₄Nradiohalide, to yield the alkyl 2′-radiohalodeoxylactose.

One embodiment of the present invention relates to a method of positron emission tomography (PET) imaging of cancer cells in a mammal to detect the levels of expression or activity of a lactose-binding protein by the cancer cells, comprising administering to the mammal a composition containing an alkyl 2′-radiohalodeoxylactose, and imaging the mammal with PET.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the synthesis scheme for ethyl 2′-fluorodeoxylactose.

FIG. 2 shows the synthesis scheme for ethyl 2′-[¹⁸F]fluorodeoxylactose.

FIG. 3 shows a chromatogram of a protected ethyl 2′-fluorodeoxylactose precursor and an identically-protected ethyl 2′-[¹⁸F]fluorodeoxylactose precursor.

FIG. 4 shows a chromatogram of ethyl 2′-[¹⁸F]fluorodeoxylactose.

### DESCRIPTION OF ILLUSTRATIVE EMBODIMENTS

“Radiohalide,” as used herein, refers to any halogen isotope which decays by emitting a positron. Examples of radiohalides include, but are not limited to, ¹⁸F, ¹²⁴I, ¹²⁵I, and ¹³¹I.

One embodiment of the present invention relates to a method of synthesizing an alkyl 2′-radiohalodeoxylactose, comprising treating an alkyl 2′-triflate deoxylactose with n-Bu₄Nradiohalide, to yield the alkyl 2′-radiohalodeoxylactose.

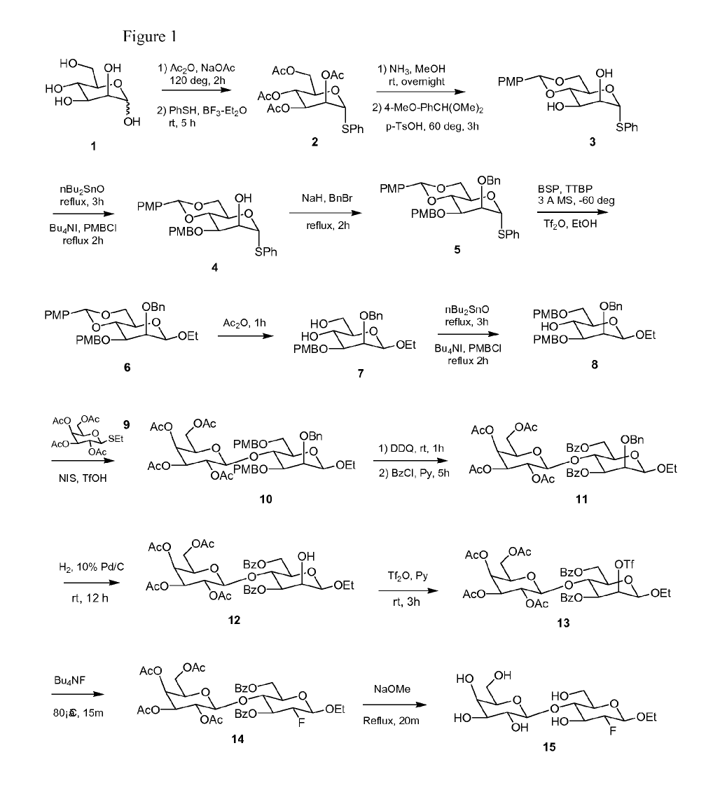

An alkyl 2′-triflate deoxylactose is a deoxylactose having an —O(alkyl) group at the 1′ position and an —O(triflate) group at the 2′ position. The alkyl 2′-triflate deoxylactose can have further substituents, such as alkyl acid ethers or aryl acid ethers at other positions. The alkyl 2′-triflate deoxylactose can be synthesized by a scheme such as that shown in FIG. 1, with routine variations in the steps, the reagents, and the reaction conditions shown therein being a matter of routine experimentation for the ordinary skilled artisan.

The treatment of the alkyl 2′-triflate deoxylactose with n-Bu₄Nradiohalide can be performed under any appropriate conditions. Anhydrous solvents, such as dry MeCN, are most useful. The treatment can be performed at any appropriate temperature, such as from about 50° C. to about 120° C., for example, at about 80° C., for a duration sufficient to produce the alkyl 2′-radiohalodeoxylactose in high yield, such as from about 5 min to about 60 min, such as for about 15 min.

In one embodiment, the method further comprises (starting with an alkyl 3′,6′-dibenzoate deoxylactose, the synthesis of which can be performed by the technique described in Example 1, below) triflation of an alkyl 3′,6′-dibenzoate deoxylactose, to yield an alkyl 2′-triflate, 3′,6′-dibenzoate deoxylactose; using the alkyl 2′-triflate, 3′,6′-dibenzoate deoxylactose as the alkyl 2′-triflate deoxylactose in the treating step, to yield alkyl 2′-radiohalo, 3′,6′-dibenzoate deoxylactose; and deprotection of the alkyl 2′-radiohalo, 3′,6′-dibenzoate deoxylactose, to yield alkyl 2′-radiohalo deoxylactose.

Triflation can be performed by the use of ditrifyl oxide (CF₃SO₂)₂O) in the presence of a suitable solvent, such as pyridine, under appropriate conditions of temperature (such as from about 0° C. to about 40° C., such as at about room temperature) and duration, to yield the alkyl 2′-triflate, 3′,6′-dibenzoate deoxylactose.

The deprotection of the alkyl 2′-radiohalo, 3′,6′-dibenzoate deoxylactose can be performed using any reactants and solvents known for use in replacing benzoate protecting groups with protons, such as NaOMe in MeOH.

In PET, a short-lived radioactive tracer isotope, such as ¹⁸F (half life≈110 min), which decays by emitting a positron, is chemically incorporated into a metabolically active molecule and injected into a living subject. Injection into blood circulation is the most common, but PET is not limited thereto. After injection, the metabolically active molecule becomes concentrated in tissues of interest that contain molecules, enzymes, or other structures which interact with the metabolically active molecule and the subject is placed in the imaging scanner. Decay of the short-lived isotope emits a positron. After traveling a short distance (typically no more than a few millimeters) the positron annihilates with an electron, producing a pair of annihilation photons (similar to gamma rays) moving in opposite directions. These are detected when they reach a scintillator material in the scanning device, creating a burst of light which is detected by photomultiplier tubes.

The most significant fraction of electron-positron decays result in two 511 keV photons being emitted at almost 180 degrees to each other, allowing localization of their source along a straight line of coincidence. Using statistics collected from tens-of-thousands of coincidence events, a set of simultaneous equations for the activity of each parcel of tissue along many lines of coincidence can be solved, and thus a map of locations and radioactivities in the body may be plotted. The resulting map shows the tissues in which the molecular probe has become concentrated, and can be interpreted by nuclear medicine physician or radiologist in the context of the patient's diagnosis or treatment plan.

One embodiment of the present invention relates to molecular probes analogous to anti-cancer drugs which target given proteins expressed by given alleles of various oncogenes, which probes can be used in PET to determine whether the given proteins are targeted by the molecular probe and hence whether the anti-cancer drug would be likely to be efficacious against the cancer characterized by activity of the given allele of the particular oncogene.

The short half-lives of many radionuclides useful in PET make necessary the rapid incorporation of the radionuclides into metabolically active molecules.

One embodiment of the present invention relates to a method of positron emission tomography (PET) imaging of cancer cells in a mammal to detect the levels of expression or activity of a lactose-binding protein by the cancer cells, comprising administering to the mammal a composition containing an alkyl 2′-radiohalodeoxylactose, and imaging the mammal with PET.

In one embodiment, the lactose-binding protein is lectin.

In one embodiment, the cancer cells are pancreatic carcinoma cells.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Lactose-binding proteins, like lectin, are overexpressed in several types of tumors, like pancreatic carcinomas, colon carcinomas. Radiolabeled lactose was synthesized as a molecular probe for the PET imaging of lactose-binding protein expression in pancreatic carcinomas. Here we describe the improved methods of cold synthesis, radiolabeling and in vivo tissue distribution in rats.

Synthesis of ethyl 2′-fluorodeoxylactose

As shown in FIG. 1, the synthesis of ethyl 2′-fluorodeoxylactose 15 was carried out as shown in Scheme starting from commercial available D-Mannose 1. Treatment of D-mannose with acetic anhydride and sodium acetate at 100-120° C. for 2 h gave pentaacetate of D-mannose quantitatively. Further treatment of pentaacetate with thiophenol, followed by BF₃.Et₂O yielded 2 in 94% yield. 2 was deprotected by treatment with 7 N NH₃ in MeOH overnight. The thus formed crude phenyl 1-thio-α-D-mannopyranoside was treated with 4-methoxybenzaldehyde dimethyl acetal, and trace amount of p-TsOH in DMF to give 3 in 65% yield. Selective protection of 3-OH in 3 was carried out by treatment 3 with Bu₂SnO, followed by PMBCl and Bu₄NI. 4 was formed in 78% yield. Further protection of 4 with NaH and BnBr in THF gave 5 quantitatively. 6 was subsequently obtained from 5 by activation with 1-benzenesulfinyl piperidine and triflic anhydride in the presence of 2,4,6-tri-butylpyrimidine in 70% yield. Treatment of 6 with glacial acetic acid gave diol 7 in 97% yield. Reaction of 7 with dibutyltin oxide in toluene at reflux, followed by treatment with PMBCl and Bu₄NI provided 8 in 80% yield. Glycosylation of 9, which was prepared from β-D-galactose pentaacetate and ethanethiol in 80% yield, with 8 in the presence of N-iodosuccinimide and TfOH gave 10 in 89% yield. Selective removal of two PMB protection group in 10 using DDQ, followed by protection by treatment with BzCl and pyridine provided 11 in 70% yield. Hydrogenation over 10% Pd/C of 11 in EtOAc-Et₂O (1:2) yielded 12 in 94% yield. Further treatment of 12 with Tf2O in the presence of pyridine at rt provided the precursor 13 in 69% isolation yield.

The precursor 13 was then converted to the corresponding fluoride 14 by treatment with Bu₄NF in CH₃CN at 80° C. for 10 min in a 10% yield after HPLC separation. The quantitative deprotection of 14 with NaOMe in MeOH completed the cold version synthesis of target compound 15.

Radiolabeling to yield ethyl 2′-[¹⁸F]fluorodeoxylactose

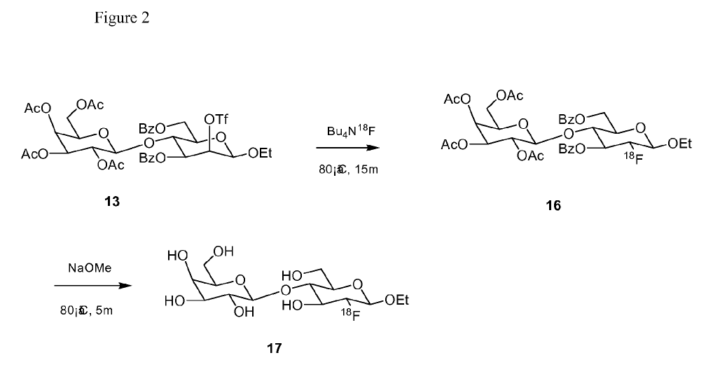

As shown in FIG. 2, radiofluorination was performed on the triflate precursor in dry MeCN using n-Bu₄N18F at 80° C. for 15 min. The reaction mixture was passed through silica gel Sep-Pack cartridge and eluted with EtOAc. Solvent was evaporated and the crude product was purified by HPLC to isolate ¹⁸F-labeled protected lactose in 65% MeCN in water. Solvent was partially evaporated and the product was passed through a reverse phase cartridge, and eluted with MeOH (2 mL) then hydrolyzed with NaOMe by refluxing for 5 min. The product was neutralized with HCl. MeOH was evaporated and re-dissolved in saline. The product was filtered through a Millipore filter for biological studies.

The radiochemical yields were 65-70% (d.c.) with an average of 68% in 3 runs. Radiochemical purity was >99% with specific activity >74 GBq/μmol at the end of synthesis. Synthesis time was 110-115 min from the end of bombardment.

Chromatography

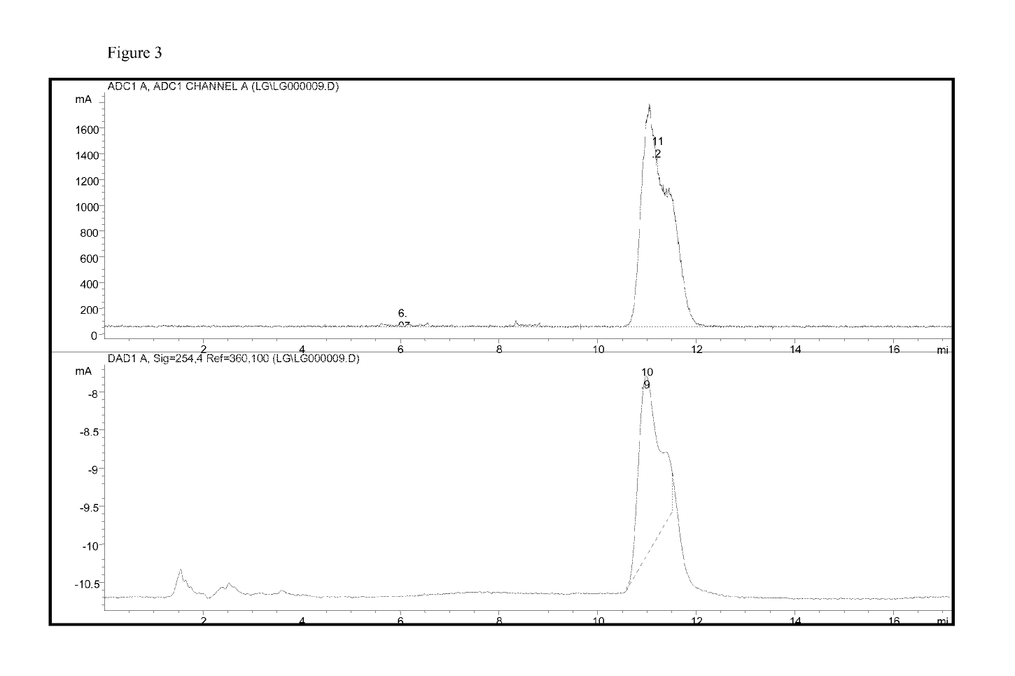

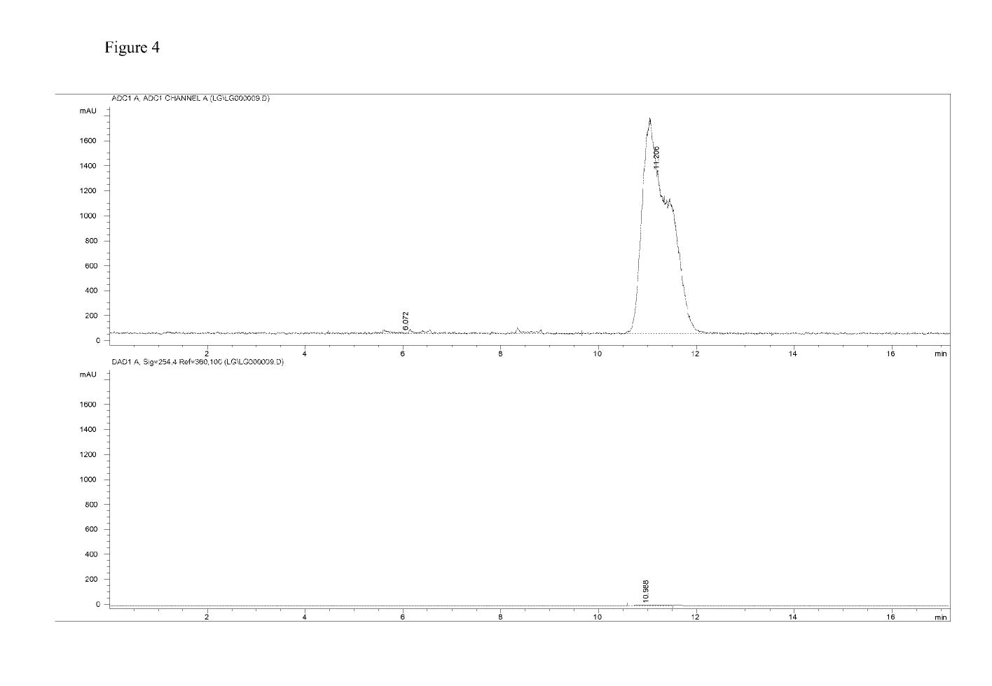

FIG. 3 shows HPLC chromatography results for coinjection of purified 16 with its cold version (14). FIG. 4 shows a chromatogram of final compound 17.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Yun, M.; Chun, K. H.; Shin, J. N.; Oh, J. Bull. Korean Chem. Soc. 2002, 23, 177-178.

What is claimed is:

1. A method of synthesizing an alkyl 2′-radiohalodeoxylactose, comprising:
treating an alkyl 2′-triflate deoxylactose with n-Bu₄Nradiohalide, to yield the alkyl 2′-radiohalodeoxylactose.

2. The method of claim 1, further comprising:
triflation of an alkyl 3′,6′-dibenzoate deoxylactose, to yield an alkyl 2′-triflate, 3′,6′-dibenzoate deoxylactose, wherein the alkyl 2′-triflate, 3′,6′-dibenzoate deoxylactose is the alkyl 2′-triflate deoxylactose in the treating step and the treating step yields alkyl 2′-radiohalo, 3′,6′-dibenzoate deoxylactose; and
deprotection of the alkyl 2′-radiohalo, 3′,6′-dibenzoate deoxylactose, to yield alkyl 2′-radiohalo deoxylactose.

3. A method of positron emission tomography (PET) imaging of cancer cells in a mammal to detect the levels of expression or activity of a lactose-binding protein by the cancer cells, comprising:
administering to the mammal a composition containing an alkyl 2′-radiohalodeoxylactose, and
imaging the mammal with PET.

4. The method of claim 3, wherein the lactose-binding protein is lectin.

5. The method of claim 3, wherein the cancer cells are pancreatic carcinoma cells.
